(19) United States
(12) Patent Application Publication
(10) Pub. No.: US 2026/0068859 A1
(43) Pub. Date: Mar. 12, 2026

(54) MEASURING SYSTEM AND METHOD FOR NON-INVASIVE IN-OVO SEXING OF A BIRD EMBRYO IN AN EGG IN EARLY EMBRYONIC DEVELOPMENT

(71) Applicant: OMEGGA GMBH, Munich (DE)

(72) Inventors: Till Nöllgen, München (DE); Paul Günther, München (DE); Moritz Eder, Dachau (DE); Kyle Hiroyasu, München (DE)

(21) Appl. No.: 19/106,203

(22) PCT Filed: Aug. 23, 2023
(86) PCT No.: PCT/EP2023/073080

§ 371 Date: Feb. 24, 2025

(30) Foreign Application Priority Data
Aug. 29, 2022 (DE) 102022121790.6

(51) Int. Cl. A01K 45/00; G01N 21/31; G01N 33/08

(52) U.S. Cl. CPC A01K 45/00; G01N 21/31; G01N 33/08; G01N 2201/021; G01N 2201/127

(57) ABSTRACT

The present invention relates to a measuring system for the non-invasive, preferably automated, sex detection of embryos in an egg (50) in early-embryonic development, in particular before the seventh day of incubation, in particular during incubation, in particular in an incubator having at least one egg tray (40) for accommodating a plurality of eggs (50) and at least one egg trolley (80) for holding at least one egg tray (40), having at least one irradiation unit (10) for irradiating an egg (50) with electromagnetic radiation; at least one sensor unit (20) for detecting electromagnetic radiation transmitted through the egg (50); an evaluation unit (30) having at least one spectrometer (31), which is designed to receive the radiation transmitted through the egg (50) and to generate a spectrum of the radiation transmitted through the egg (50); and a data processing unit (33), which is designed to receive spectra generated by the spectrometer (31) and to store them as a reference spectrum or measured spectrum; an identification unit (23) for generating identification data, by means of which the spectra generated on one egg (50) can be uniquely associated with the egg (50); and a classification unit (35); wherein the data processing unit (33) is designed to store spectra generated by the spectrometer (31) and associated identification data, and wherein the classification unit (35) is designed to determine the sex of the embryo based on at least one reference spectrum and at least one measured spectrum. A corresponding method is further specified within the scope of the invention.

The present invention relates to a measuring system for the non-invasive, preferably automated, sex detection of embryos in the egg in early embryonic development, in particular before the seventh day of incubation, in particular during incubation, according to the subject matter of claim 1, as well as a corresponding method according to the subject matter of claim 19.

Systems for detecting the sex of an embryo have been in use for some time in the commercial cultivation of livestock, for example, chicks. As it is desirable that the embryos, especially the female embryos, are not thereby damaged, in order that further cultivation is not endangered and also to minimize consumables and mechanical effort, non-invasive devices and methods are thereby particularly advantageous.

A commonality of most of these devices is that they determine the sex of the embryo using so-called “candling”. Radiation is thereby passed through the egg. Information about the state of the egg or the embryo is collected based on the radiation emerging back out of the egg.

However, due to the large biological variance in the properties of the egg, such as size, shape, color and shell thickness, and the associated wide range of possible measurement results, it is difficult to make reliable quantitative statements. These factors make sex determination considerably more difficult, particularly in the early stages of development of the embryo, in which the signals sought are still quite weak.

A reliable means for detecting the sex of an embryo at an early stage is necessary. Particularly due to ethical concerns and the resulting legal requirements, it is thereby essential to carry out sex detection as early as possible, especially before the development of a sense of pain (7th day of incubation), in order to both reduce animal suffering in the breeding of laying hens, as well as to offer hatcheries a cost-effective alternative for the resource-and cost-intensive raising of male, laying hen chicks, which, due to their different meat consistency, may only be used as a niche product and may find no buyers.

As, in addition, large quantities of eggs to be determined arise in the commercial cultivation of eggs, it is also difficult to provide measuring systems with the ability to enable a correspondingly high throughput without generating excessive costs, due to the large amounts of sensor technology required.

Another difficulty is the frequently encountered requirement to provide measuring systems that are compatible with already existing incubators and the egg trays, which are used to store and incubate eggs, in order to avoid new purchases as much as possible.

In light of the preceding statements, the object of the present invention is to provide a system and a method which allow large quantities of eggs to be examined to determine their sex, wherein a high level of accuracy is achieved early in the incubation cycle. Furthermore, it should be possible to realize a high degree of integration into already existing cultivation systems.

This problem is solved by a measuring system having the features of claim 1, and also a method having the features of claim 19. The subclaims specify preferred further refinements.

The problem is solved, in particular, by a measuring system for the non-invasive, preferably automated, sex detection of embryos in an egg in early embryonic development, in particular before the seventh day of incubation, in particular during incubation, in particular in an incubator having at least one egg tray for accommodating a plurality of eggs and at least one egg trolley for holding at least one egg tray, having:
at least one irradiation unit for irradiating an egg with electromagnetic radiation;
at least one sensor unit for detecting electromagnetic radiation transmitted through the egg;
an evaluation unit with
   at least one spectrometer, which is preferably connected to the sensor unit, and which is designed to receive the radiation transmitted through the egg and to generate a spectrum of the radiation transmitted through the egg; and
   a data processing unit which is designed to receive spectra generated by the spectrometer and to store them as a reference spectrum or a measured spectrum;
an identification unit for generating identification data, by means of which the spectra generated on an egg are uniquely assignable to the egg;
a classification unit;
   wherein the data processing unit is designed to store spectra generated by the spectrometer and associated identification data, and wherein the classification unit is designed to determine the sex of the embryo based on at least one reference spectrum and at least one measured spectrum.

One essential idea of the invention is that an individualized reference spectrum is assignable to an individual egg, said reference spectrum may be used to take disruptive factors, caused by the biological variance, into account for a measurement, and thus to enable a more precise determination of the sex of the embryo at an early point in time.

Transmission data is thereby generated from (electromagnetic) radiation transmitted through the egg, which is compiled into a spectrum. Conclusions may be drawn about spectral absorption ranges in the egg, based on the detected spectrum, which may hereby be composed of a single measurement or multiple measurements of the radiation transmitted through the egg (transmission data). The information generated by the data processing unit is thereby forwarded to the classification unit, which carries out the final classification.

The components of the evaluation unit may be provided by separate units that are communicatively connected to one another. Likewise, multiple or all units of the evaluation unit may be provided as a common structural unit. The data processing, identification, and classification units may be formed by microprocessors or software components, which are stored on a computing device and are executable there in order to provide the claimed functions.

According to one preferred embodiment, the measuring system has an optical decoupling element for optical decoupling of the irradiation unit and the sensor unit, said optical decoupling element preferably being designed to contact the egg during a measurement.

A substantial disruptive factor when measuring a spectrum, in particular a transmission spectrum, is scattered light, which enters the sensor without first passing through the egg, as this is of high intensity but does not carry any relevant spectral information. In order to prevent this, an optical decoupling element is used, which prevents light from directly entering into the sensor from the irradiation unit. Such a decoupling element may thereby have the form of a screen or a brush, which contacts the shell of the egg as closely as possible in order to leave few possible gaps open for the exit or entry of scattered light. It is thereby understood that, when using radiation outside the visible range, a corresponding sealing screen may be used.

The decoupling element may be designed and arranged in such a way that it shields the at least one irradiation unit from the environment. In particular, the decoupling element may be arranged and designed in such a way that an egg may be arranged on the decoupling element in such a way that radiation, emitted by the irradiation unit, is essentially completely directed to the egg. Likewise, the decoupling element may be designed and arranged in such a way that it shields the at least one sensor unit from the environment. In particular, the decoupling element may be arranged and designed in such a way that an egg is is arrangeable on the decoupling element in such a way that the sensor unit is shielded from the environment of the egg.

The measuring system preferably has means for determining an angle between a reference axis of the egg and a reference axis of the measuring system. A stationary reference axis, for example the vertical (i.e., a direction parallel to the direction of gravity), may function as the reference axis for the measuring system. An axis, that runs through the two tips of the egg, i.e., an axis to which the shell of the egg is essentially rotationally symmetrical, may be selected as the reference axis of the egg.

Since, in the first few days of development, the embryo will align itself in each position of the egg so that it floats at the top, knowing a tilt angle of the egg or of the optical measuring axis relative to a reference axis of the measuring system is beneficial in order to ensure where the embryo is located in relation to the visual field of the sensor unit. Furthermore, the measurement at different tilt angles leads to an increase in the variance of the detected spectra. The knowledge of the tilt angle of the egg may additionally be used to improve a later comparability of the reference spectra with the measured spectra, for example, in that the embryo is calculated out by combining different measurements at different tilt positions.

It is hereby particularly preferred that the reference axis of the measuring system is formed by a stationary axis, for example, the vertical, which is unchangeable even during a movement of the measuring system. If the measuring system is designed in such a way that the position and orientation of the egg may be determined relative to the radiation source and the sensor unit, a tilting of the egg, together with the radiation source and the sensor unit, does not lead to a change in the relative position and orientation of the egg with respect to the radiation source and the sensor unit. Nevertheless, the position of the embryo changes relative to the radiation source and sensor unit.

The tilting may also be determined in this case, when a stationary axis, for example, the vertical, is selected as the reference axis of the measuring system. A gyroscope, which is preferably rigidly connected to a component of the measuring system, may be used, for example, as a means for determining the angle between the reference axis of the egg and a stationary reference axis of the measuring system, such as the vertical.

Since the embryo floats during development, but often grows decentrally on the eggshell in the course of development, and is therefore rarely positioned exactly in the middle so that it lies on the axis of symmetry of the egg, it is preferred to be able to carry out the irradiation of the egg from different directions.

It is hereby preferred that the irradiation unit has a plurality of radiation sources. The irradiation unit is particularly preferably formed by a ring light, particularly preferably by a ring LED, in which a plurality of radiation sources, preferably LEDs, are arranged in a ring. The diameter of the ring light is preferably selected such that it is smaller than the diameter of an egg at its thickest point perpendicular to the axis of symmetry of the egg. Such a radiation source, designed as a ring light, may be arranged at the blunt end of the egg and allows an irradiation of the egg from different positions or directions that are arranged radially around the axis of symmetry of the egg.

The ring light preferably has exactly or at least 4 radiation sources (for example LEDs), more preferably exactly or at least 8 radiation sources, which are preferably arranged uniformly distributed along the circumference of the ring light. The use of beam limiting devices around the individual radiation sources may be helpful in order to clearly differentiate the different irradiated areas of the egg from one another. The radiation sources of the lighting unit are preferably designed to be controllable independently from one another in order to enable a selective irradiation of the egg from different directions.

According to one further embodiment, the irradiation unit has a plurality of optical light guides, which are designed to be arranged along a ring at one end of the egg, and which are controllable independently from one another in order to guide the radiation emitted by the irradiation unit from different directions to the egg.

The sensor unit remains either statically positioned at one of the two poles or is positioned on the opposite side of the active lighting direction. In any case, it is preferred that the optical decoupling element is arrangeable on the egg in a lightproof manner.

The measuring system is preferably designed to carry out a plurality of measurements, in which the egg is illuminated from different directions in that individual radiation sources are activated sequentially, or the radiation emitted by the irradiation unit is guided sequentially through different optical light guides to the egg.

By comparing the measurements, carried out sequentially, with one another, a measurement may be identified in which the embryo is irradiated the most, i.e., a measurement with a relatively highest and/or strongest useful signal. Part of the remaining measurements or all of the remaining measurements, in which the useful signal was smaller and/or weaker, may be calculated using the measurement with the relatively highest and/or strongest useful signal (for example by means of division or subtraction), in order to improve the quality of the measurement and to improve the measured spectrum generated therefrom.

According to one preferred embodiment, the irradiation unit and the sensor unit are arranged and configured in such a way that, during each measurement, they have the same orientation to one another relative to two reference points of the egg, for example, the center of gravity of the shell of the egg or the two poles or tips of the egg.

It is advantageous to ensure that the elements of the measuring system, in particular the irradiation unit and the sensor unit, are substantially always located in the same relative arrangement and orientation to one another and to the egg, as the variance between the measurements may be significantly reduced hereby. In connection with the determination of the angle of an egg axis, targeted measurements may additionally be carried out from different angles.

One further embodiment of the present invention includes a trolley transport device for transporting an egg trolley having at least one egg tray to at least one irradiation unit, said trolley transport device being preferably arranged within the incubator, more preferably between different incubators and/or a trolley positioning device, which is suitable for uniquely determining a position of the trolley within the incubator. In this case, it is preferred that the at least one irradiation unit and the at least one sensor unit are mounted on a measuring column which may be stationarily installed in the incubator. As will subsequently be described in greater detail, the at least one irradiation unit and the at least one sensor unit may be fixedly or movably mounted on the measuring column.

Since so-called egg trolleys are generally used in commercial egg cultivation in order to accommodate a large number of egg trays and hold them within an incubator, it is advantageous if a fully automated or at least a partially automated transport of the trolleys to the irradiation and/or sensor unit may be carried out. This offers the advantage that the measuring system may be used in already existing incubators without having to replace the already existing equipment. In this case, solutions are particularly available in which the transport device already knows the position of all trolleys within the incubator in order to then approach them in a targeted manner. For this purpose, the trolley transport device may be designed as a robot, which has a control unit which is designed to steer the trolley transport device to predetermined positions within the incubator in order to transport egg trolleys from previously determined positions to the irradiation and/or sensor unit for measurement.

In addition, precise knowledge of the position of the trolleys or the trolley positioning device is also helpful in order to enable the identification of individual trolleys, egg trays or eggs for the purposes of measurement, further observation, or screening. The trolley transport device is preferably communicatively connected to the evaluation unit and/or the identification unit and is designed to transmit trolley identification data to the evaluation unit and/or to the identification unit, which are taken into account during the generation of the identification data.

Furthermore, the problem of the invention is solved by a measuring system having transport means for transporting the irradiation unit and the sensor unit to an egg, preferably within the incubator, more preferably between different incubators.

Additionally or alternatively to the previously described concept according to the invention, which transports the egg trolleys to the irradiation and/or sensor unit in order to carry out measurements on the eggs, it is likewise within the meaning of the present invention to configure the measuring system such that the irradiation unit and the sensor unit transportable to a (designated) egg tray. This is therefore particularly advantageous because in this embodiment the eggs are less disturbed in their incubation by the measurements and are therefore exposed to a lower risk of being damaged or dying during the development process.

According to one aspect of the present invention, a measuring system is provided in which the trolley transport device has means, which are designed to adjust a tilt position of at least one egg.

In order to enable the easiest compatible adjustment of the tilt position of at least one egg or the angle of the reference axis of the egg using already existing trolley elements, it is advantageous to equip the trolley transport device with additional or interacting components in order to achieve high compatibility and thus to achieve cost savings. For this purpose, it is preferred that the trolley transport device has means for adjusting the tilt angle of the egg trays in an egg trolley. The means for adjusting the tilt angle of the egg trays in an egg trolley are preferably designed to engage and/or interact with a tilting device of the egg trolley in order to adjust the tilt angle of the egg trays in the egg trolley. For example, the trolley transport device may be designed to tilt the egg trolley as a whole with the egg trays and eggs located in it, or to engage in an existing tilting device of the egg trolley to tilt the egg trays.

One further development of the present invention comprises a measuring system having a measuring arm, which accommodates the (at least one) irradiation unit and the (at least one) sensor unit in such a way that at least one egg is positionable for measurement at the (at least one) irradiation unit and/or at the (at least one) sensor unit.

A mutual accommodation of the irradiation and sensor units in one measuring arm has the advantage that this facilitates the relative positioning of the two units to one another and relative to the egg. Furthermore, an adaptation of the measuring device to an angle of an egg tray is significantly facilitated, as only the angle and the position of the measuring arm need to be adjusted.

According to one further aspect of the present invention, the measuring system includes a first measuring arm, which accommodates the (at least one) irradiation unit, and a second measuring arm, which accommodates the (at least one) sensor unit, wherein the first measuring arm and the second measuring arm are arranged and designed in such a way that at least one egg is positionable for measurement between the (at least one) irradiation unit and the (at least one) sensor unit.

This design enables an arrangement of the irradiation unit relative to the sensor unit at opposite ends of the egg. This has the advantage that the measuring system may be equipped to measure the eggs by passing radiation through them. By this means, the luminosity ay be increased in comparison to detecting radiation scattered back or to the side.

In one further aspect of the present invention, the measuring system has the following: a measuring column on which the first measuring arm and the second measuring arm are movably mounted, wherein the first measuring arm has a first movement mechanism for adjusting a vertical position of the first measuring arm, and wherein the second measuring arm has a second movement mechanism for adjusting a vertical position of the second measuring arm.

A movable arrangement of the measuring arms thereby enables a measurement at a plurality of egg trays without requiring a separate measuring arm for each level. By this means, the number of irradiation and sensor units required overall may be kept low. This enables, among other things, easy integration into already existing incubation devices.

One further possible embodiment has a measuring system according to the invention, wherein the first movement mechanism has a first horizontal linear guide and a second horizontal linear guide for adjusting a horizontal position of the first measuring arm, and wherein the second movement mechanism has a third horizontal linear guide and a fourth horizontal linear guide for setting a horizontal position of the second measuring arm.

Using this type of a linear guide allows the irradiation unit and the sensor unit to be freely moved and positioned relative to one another. By this means, an easy possibility is created for changing between different egg trays, which both increases the measurement throughput and also increases the compatibility, and thus saves costs.

Furthermore, a possible embodiment of the present invention comprises a measuring attachment, which accommodates both the irradiation unit and also the sensor unit, so that both the irradiation unit and also the sensor unit may be arranged either above or below the egg tray, in particular for a measurement. The measuring attachment is configured to be placed on the egg tray during the measurement. For this purpose, the measuring attachment may have a measuring attachment adapter, which enables a fixed positioning of the measuring attachment relative to an egg tray.

This type of measuring attachment has the advantage that it may be placed on egg trays that are already in use without any major complications. This is particularly advantageous because high retrofitting costs are thereby avoided. In addition, the attachment may be easily moved back and forth between different egg trays, egg trolleys and also incubators, both manually and also by an automated transport device. In addition, this type of attachment automatically adjusts to a changeable angle of the egg tray without requiring a new alignment.

In one preferred embodiment of the invention, the measuring system comprises at least one ventilation opening, which is formed in the measuring attachment, in the first measuring arm and/or in the second measuring arm, and is designed to ensure ventilation of the egg during a measurement.

For optimal incubation conditions for the eggs, it is necessary to maintain as large an area of the eggs as possible in constant heat exchange with the ambient air of the incubator. A ventilation duct according to the invention thus ensures that a constant flow of incubator air is in heat exchange with the surface of the egg even during the measurement. A ventilation duct may thereby be understood to be all openings that allow the incubator air to come into contact with the egg during the measuring process. In particular, the ventilation opening may be formed by a porous or honeycomb structure in order to provide the greatest possible air exchange.

In one further preferred embodiment of the present invention, the evaluation unit is designed to output a sex label and an associated confidence level for each egg. This may also be output to the classification unit.

As the identification of the sex of an embryo may not be possible with absolute reliability, especially in the early incubation phase, it is of great advantage to provide a confidence level to the division of the eggs into the two sexes. The confidence level may then be used, for example, as a selection criterion. For example, the expected value of a certain number of male or female embryos may be maximized or minimized, or a predetermined probability for a predetermined minimum number of female embryos may be determined. The confidence level may be generated in the classification process by various algorithmic methods based on measurement and reference spectra. It depicts how clearly the division into the sex is to be assessed and is therefore a measure of uncertainty.

In one further embodiment, the measurement system may have an (externally connected) data memory, in particular a cloud storage, which is configured to store external parameters, such as the mortality rate of the embryos or the desired output quantity, measurement and reference spectra, and/or results of the evaluation of the measurement system, and to output to the evaluation unit or to the classification unit.

This has the advantageous effect that selection decisions may be easily made, which, in particular, also take into account the measurement results of other trolleys or incubators in order to sensibly control the overall output quantity. The external parameters may be specified and adjusted by the operator of the incubator.

According to one further embodiment of the present invention, the measuring system comprises fixing means, which are designed to prevent a change in the orientation of the egg in relation to the egg tray.

Since the angular position of the egg has a large influence on the measured spectra due to the floating of the embryo, fixing this angle allows greater reproducibility and accuracy to be achieved in all measurements, as well as when presetting or changing the angle. The accuracy and reliability of the sex determination is thereby further improved. The fixing means may thereby be both a component of one of the measuring arms, or of the measuring attachment, or of the measuring attachment adapter, and also a separate component of specific egg trays, provided for this purpose as part of the measuring system.

One further embodiment according to the invention comprises the above measurement system, wherein the evaluation unit has a classification unit which, based on the data from the measuring unit and/or from the externally connected data memory, carries out a classification of an egg according to sex and/or health status, wherein the classification unit is preferably spatially separated from the rest of the evaluation unit. In particular, the classification unit may be formed by a software component on an external server, preferably on a cloud server.

Separating the evaluation and classification units may facilitate a control to be assumed for the output of multiple trolleys or incubators. In this case, only pre-processing of the measured data on site is necessary, while the actual selection decisions are carried out spatially and/or temporally separated due to larger amounts of data.

The problem of the invention is further solved by a method for the non-invasive sex detection of an embryo in an egg in early embryonic development, in particular before the seventh day of incubation, in particular during incubation, having the following steps:
Generating at least one reference spectrum before and/or at the start of incubation by means of irradiating the egg with electromagnetic radiation, and detecting radiation that has passed through the egg, generating identification data for uniquely identifying the egg and storing the reference spectrum together with the identification data of the egg;
Generating at least one measured spectrum during incubation by means of irradiating the egg with electromagnetic radiation and detecting radiation that has passed through the egg;
Evaluating the measured spectrum using the stored reference spectrum associated with the egg to determine the sex of the embryo.

In the method according to the invention, the reference spectrum and/or the measured spectrum may be generated from a plurality of individual measurements, preferably from 10 measurements or more, more preferably from 30 measurements or more.

By combining different measurements, the reliability of a spectrum increases due to the statistical suppression of randomized disruptive factors.

One further refinement of the method according to the invention consists in that an exposure time of less than 60 μs, preferably less than 40 μs, more preferably less than 20 μs is used for generating a measured or reference spectrum.

On the one hand, this is an advantageous because, during a possible plurality of measurements on a very large number of eggs, a large savings in time is generated. On the other hand, a short exposure time minimizes the health risks for the embryo.

According to one further idea of the invention, an angle is determined between a longitudinal axis of the egg and a reference axis of the measuring system. A stationary axis, for example, the vertical may in turn function as the reference axis of the measuring system. An axis, that runs through the two tips of the egg, i.e., an axis to which the shell of the egg is essentially rotationally symmetrical, may be selected as the reference axis of the egg.

Since the embryo floats at the top relative to the direction of gravity in the early incubation phase, a determination and/or adjustment of the tilt angle of an egg allows a desirable positioning of the embryo relative to the egg shell. Furthermore, by determining the angle between the reference axis of the egg and the measuring axis of the measuring system, the variances in the measurements caused by varying tilt angles of the measuring system including the egg may be significantly reduced. Knowledge of the tilt angle of the measuring system including the egg may additionally be used in order to improve a later comparability of the reference spectra with the measured spectra.

According to one further possible embodiment of the present invention, after a measurement has been carried out, the angle of the egg (thus, the angle of the longitudinal axis of the egg relative to a reference axis of the measuring system) is changed, and, after the egg has reached a state of equilibrium, a further measurement is carried out using the changed angle of the egg. To achieve the state of equilibrium after a change of the angle of the egg, a predetermined waiting period, e.g., at least 5 seconds or at least 10 seconds, may be waited out before another measurement is carried out using the changed angle of the egg.

This may be particularly advantageous for the purposes of a reference measurement, for example, in order to tilt the embryo out of the image area of the sensor unit, which enables a greater contrast to later measurements with the embryo. This increases the accuracy of the sex determination, especially in the early phases of embryonic development.

It is further preferred that a plurality of measurements is carried out in which the egg is irradiated from (at least) 4 different directions, preferably (at least) 6 different directions, more preferably from 8 or more different directions to generate a reference spectrum and/or a measured spectrum. A measurement is preferably carried out for each different direction of the irradiation in order to obtain different spectra for different irradiation directions, said spectra may be processed into a reference spectrum or a measured spectrum.

This may be achieved, for example, by using an irradiation unit having a plurality of radiation sources, particularly preferably by using a ring light, in which a plurality of radiation sources is arranged in a ring-shaped irradiation unit along the circumference of the ring, and may be controlled or activated independently of one another. It is also conceivable to change the orientation of the irradiation unit relative to the egg.

Furthermore, the present invention comprises a preferred method, wherein sequential measurements are carried out while irradiating the egg from different directions, and the respective strength of a useful signal is determined in the measurements. One or more measurements with the strongest and/or highest useful signal is/are preferably identified on the basis of the respective strength of the useful signal.

This is particularly advantageous, as the embryo does not always float centrally at the top of the egg, but instead is often laterally offset from the central axis of symmetry of the egg and grows decentrally in the later stage of development. The signal quality (and thus the reliability of the sex determination) may thus be improved by selecting a measurement with the comparatively strongest and/or highest useful signal.

Furthermore, it is preferred that the measurement with the strongest or highest useful signal is calculated using one or more measurements with a weaker or lower useful signal in order to obtain an (optimized) reference or measured spectrum. The calculation may preferably include division, subtraction and/or averaging of various measurements.

For example, any subset of the plurality of measurements with different directions of illumination may be calculated or compared with one another. For example, all measurements, except the one with the strongest useful signal, may be averaged, and the result may be calculated using the measurement with the strongest useful signal. The calculation may be carried out in such a way that the measurement with the strongest useful signal is divided by the (averaged) measurement with a weaker useful signal, or in that the (averaged) measurement with a weaker useful signal is subtracted from the measurement with the strongest useful signal. The measurement with the strongest useful signal may be determined, for example, based on the absolute absorption in a specific spectral range. This increases the signal reliability and the reliability of the measurements.

Furthermore, the present invention comprises a method in which a normalization of a measured spectrum is carried out based on a stored reference spectrum, wherein the reference spectrum is preferably generated before the incubation, more preferably inside of the incubator, more preferably before reaching the incubation temperature. The normalization may be carried out, for example, by means of subtraction or division.

Developmental differences in the embryo may be better detected due to an early reference measurement. This is also particularly advantageous for an earliest possible identification of the sex of a given embryo.

In one further aspect of the present invention, the method comprises a calibration measurement for calibrating the sensor unit, wherein the calibration measurement is carried out while the sensor is covered and/or on a reference object, for example, on a Teflon reference block, and wherein the calibration measurement is preferably carried out automatically.

The accuracy of the measurements may be greatly increased by calibrating the sensor. Covering the sensor thereby enables an effective measurement of the so-called “dark noise” behavior of the detector, i.e., a measurement of events, without causal external input. The use of Teflon reference blocks offers a particular advantage, in that Teflon only causes a known attenuation of the amplitude of incoming radiation, and thereby leaves the spectral distribution essentially invariant, which may likewise be useful for calibrating the sensors.

According to one further aspect of the invention, to determine the sex of the embryo, a spectral range is used for the reference and measured spectra in a wavelength range between 520 nm and 580 nm, preferably between 540 nm and 575 nm, more preferably between 520 nm and 680 nm, more preferably between 520 nm and 870 nm. This spectral range may also be used as a useful signal in the above-described evaluation of sequential measurements using irradiation from different directions.

The selection of the relevant frequency range facilitates the sex detection, particularly due to a more precise resolution of discriminative absorption ranges. In particular, the hemoglobin absorption spectrum is of particular relevance in detecting the sex of the embryo. In principle, however, the radiation range is not limited to the visible spectrum, but instead may also include, in particular, the infrared and ultraviolet ranges.

According to one further possible development, the method comprises the inclusion of additional data, in particular from a decentralized data cloud, wherein the evaluation of the measured spectra is carried out taking the additional data into account.

The consideration of such additional data also enables, in particular, the inclusion of data, which does not originate from the actual measurement, into the classification and selection processes.

According to one idea of the invention, a confidence level is assigned to the determination of the sex of the egg.

Reference is made again at this point to the fact that the features and advantages described in the context of the measuring system according to the invention also apply and are transferable to the method according to the invention. Likewise, the described features and advantages of the measuring system, in particular the details of its composition, are also applicable to the method. Functional features that were described in the context of the measuring system according to the invention may be used as method steps in the method according to the invention. Likewise, method steps described in the context of the method according to the invention may be used in the measuring system in that corresponding components of the measuring system are designed to carry out the method steps according to the invention.

One preferred refinement of the method according to the invention is a method, wherein the confidence level of the sex determination is taken into account in order to decide about a screening of eggs based on a plurality of adjustable external parameters, such as the incubation cycle-specific death rate of the embryos, the desired output quantity, or the desired sex distribution.

In one further preferred aspect of the invention, an observation period is determined, based on the confidence level, and/or further measurements are carried out in order to increase the confidence level.

Thus, measures may be taken to increase the confidence level. This is particularly advantageous in the early embryonic phase, as the sex determination here is still particularly challenging and often error-prone.

According to one further possible development, the method comprises the simultaneous measurement of a plurality of eggs, in particular the simultaneous generation of a plurality of reference and/or measured spectra on a plurality of eggs, preferably by means of a plurality of measuring systems.

In order to guarantee that a large number of eggs are processed as efficiently as possible, and to avoid disruption of the eggs due to repeated or long-term measurements, it is advantageous to allow measurements to take place as simultaneously as possible. This is enabled in that a plurality of measurements is undertaken at the same time. In particular, this may also include a plurality of measurements on one egg, for example, using different sensors or different types of radiation.

One further (preferred) development of the method according to the invention lies in the simultaneous measurement of a plurality of eggs, carried out in such a way that the interference between different measuring systems is minimized during the generation of the plurality of reference and/or measured spectra.

This prevents unnecessary interference with a measurement due to measurements running in parallel, and thus increases the accuracy of the individual measurements. At the same time, this reduces the requirements for shielding the measuring unit from interference, which results in both a simpler and also a more cost-saving design of the entire system.

The invention is also subsequently described with regard to further features and advantages by way of exemplary embodiments, which are explained in greater detail with reference to the figures. As shown in:

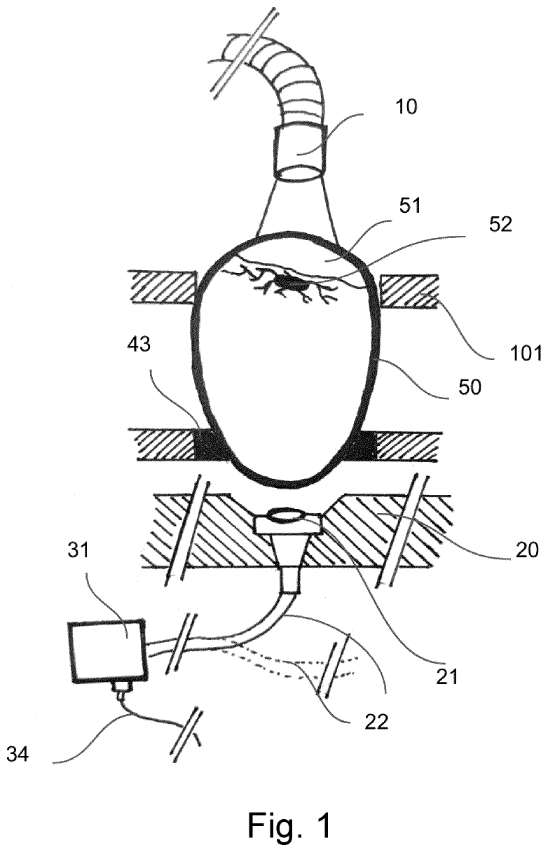

FIG. 1 a measuring arrangement of a measuring system according to one exemplary embodiment of the present invention with separate irradiation and sensor units;

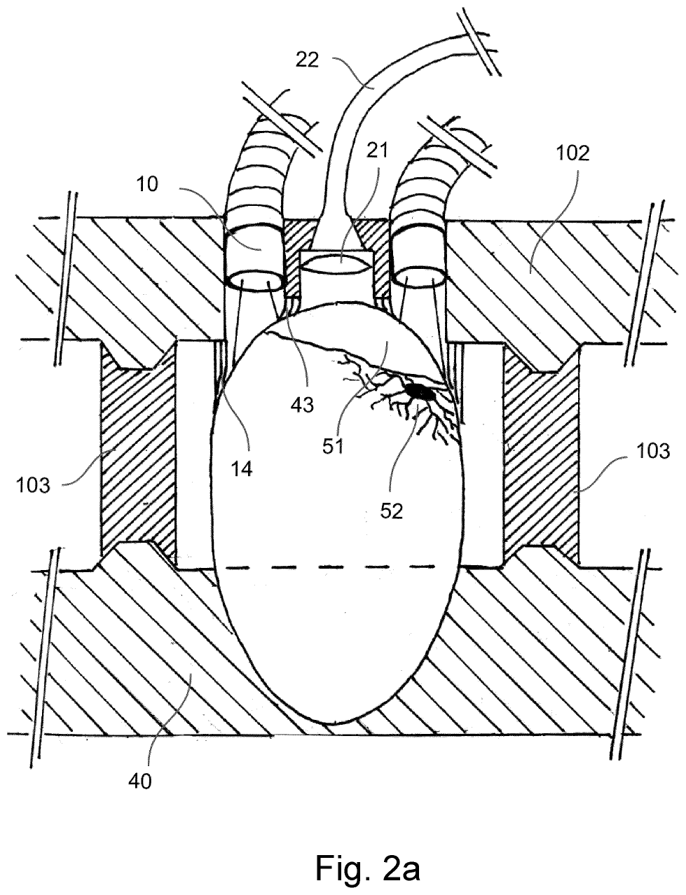

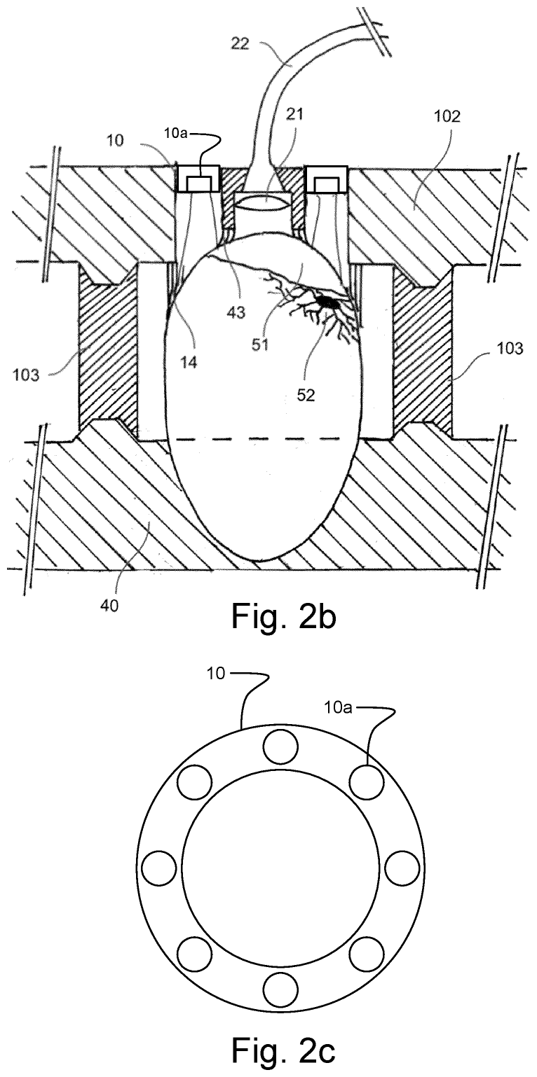

FIG. 2a a measuring arrangement of a measuring system according to one exemplary embodiment of the present invention with combined irradiation and sensor units;

FIG. 2b a variation of the measuring arrangement from FIG. 2a having an irradiation unit with a plurality of radiation sources;

FIG. 2c a view of the irradiation unit from FIG. 2b from below;

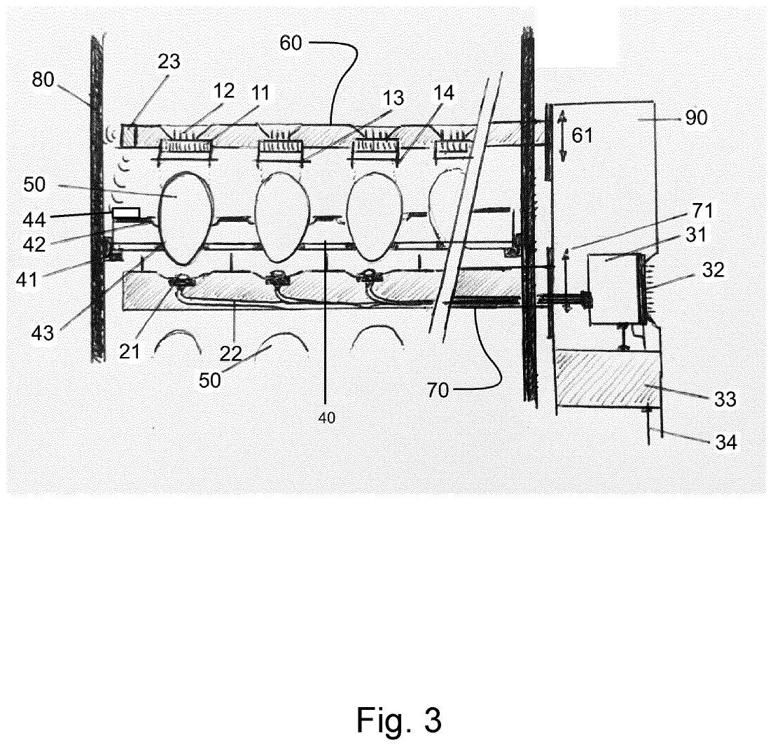

FIG. 3 a measuring system according to one exemplary embodiment of the present invention having multiple irradiation and sensor units;

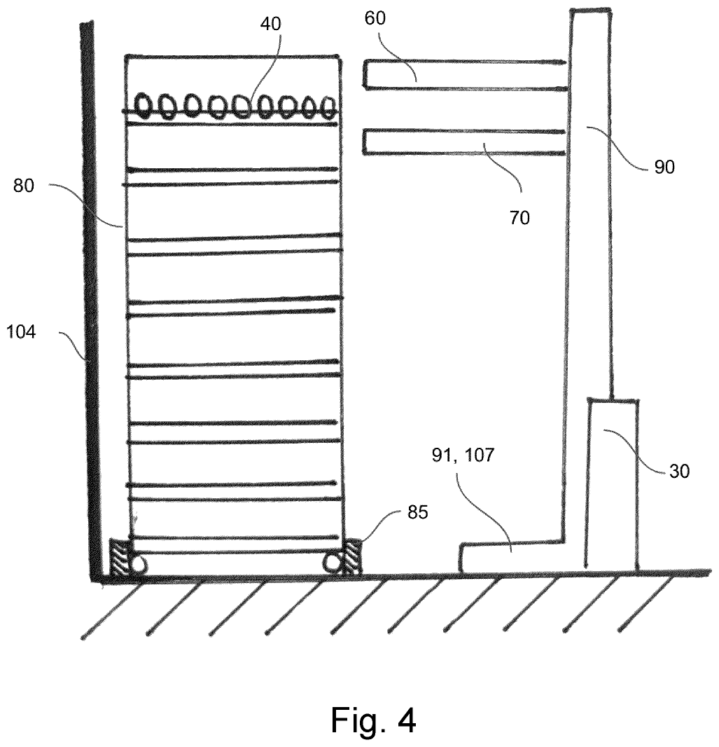

FIG. 4 a measuring system according to one exemplary embodiment of the present invention having a trolley and multiple measuring arms;

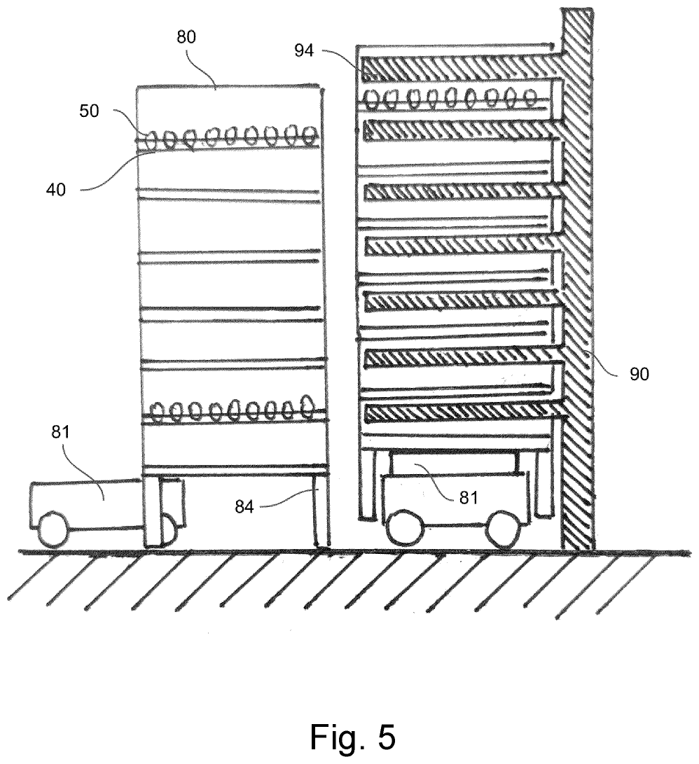

FIG. 5 a trolley transport device and also a stationary measuring unit;

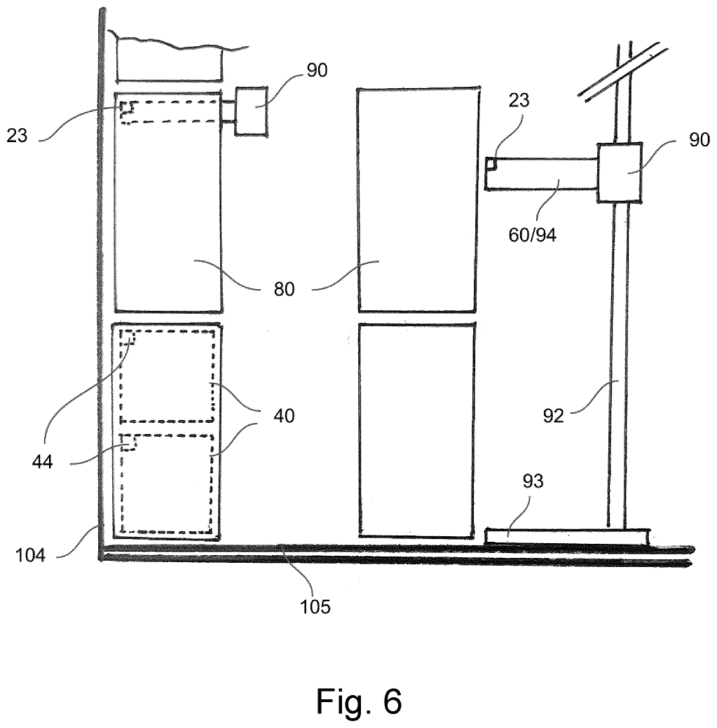

FIG. 6 a measuring system according to one exemplary embodiment of the present invention from a bird's eye view;

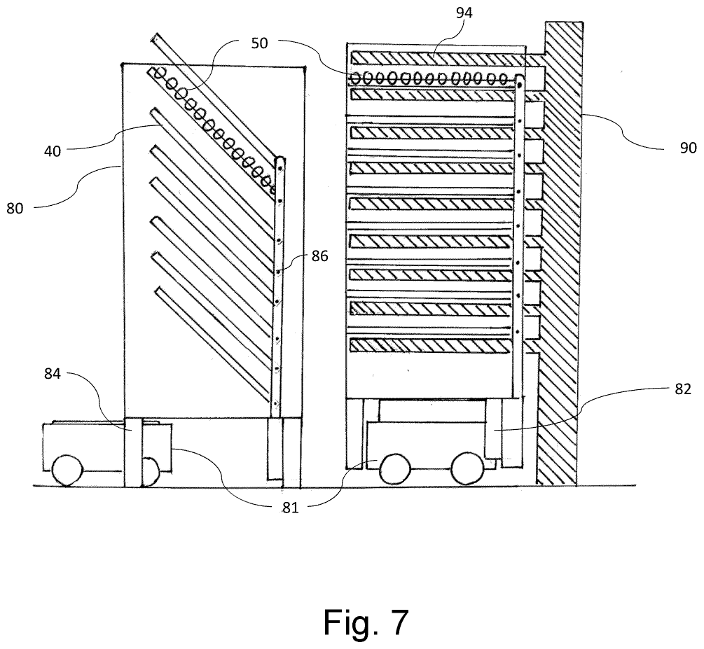

FIG. 7 a measuring column of a measuring system according to one exemplary embodiment of the present invention;

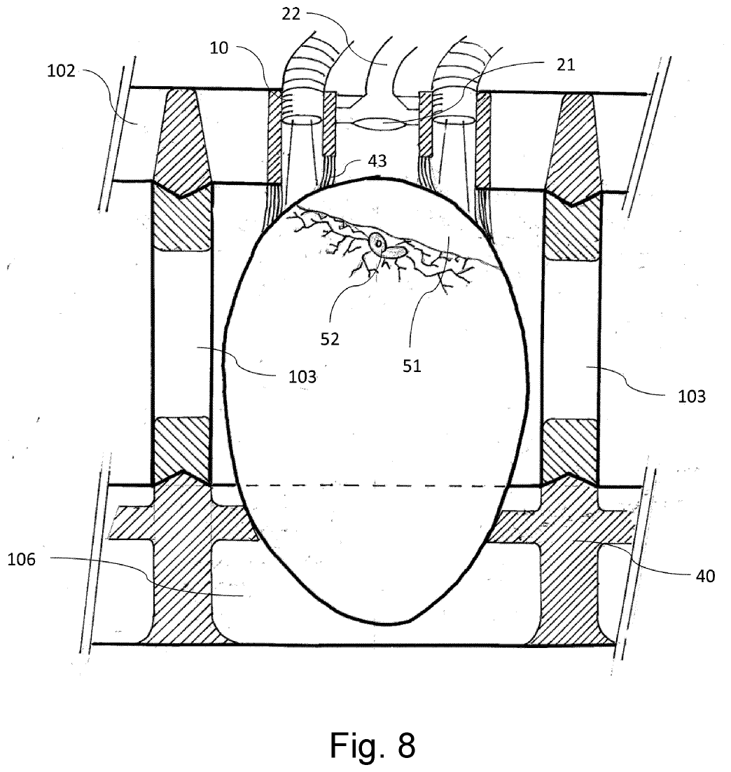

FIG. 8 a further exemplary embodiment of the measuring arrangement of a measuring system according to the invention;

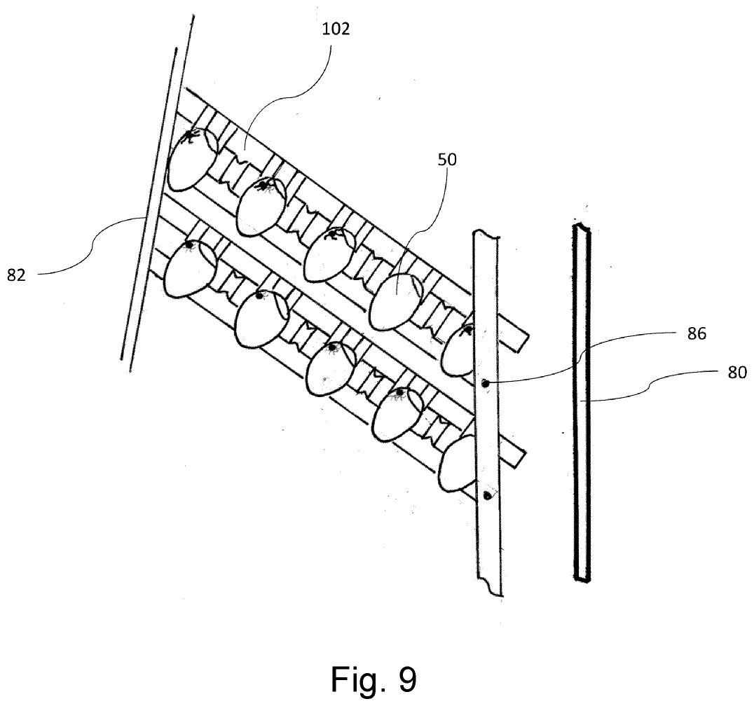

FIG. 9 a detailed view of a trolley tilting device of a measuring system according to one exemplary embodiment of the present invention;

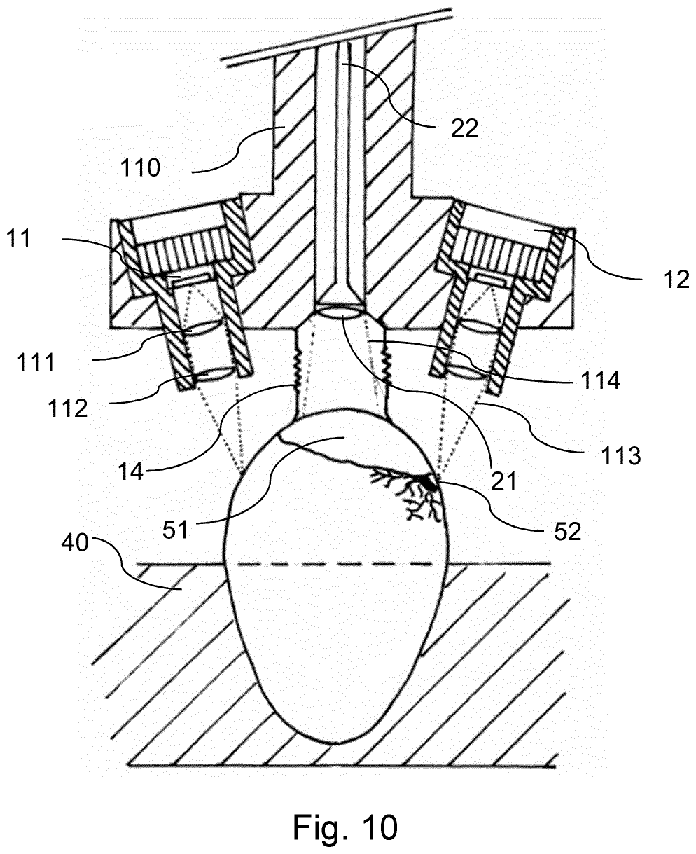

FIG. 10 a view of one further exemplary embodiment of the irradiation and sensor units;

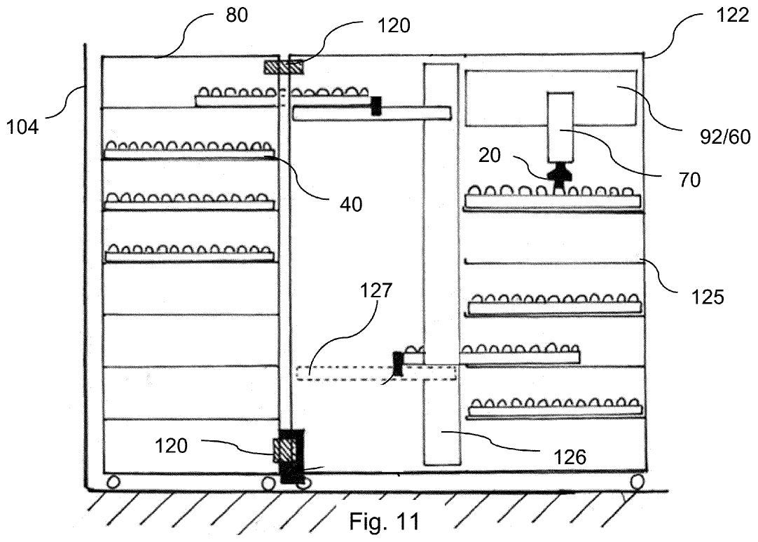

FIG. 11 one exemplary embodiment of a trolley transport device and a measuring unit.

The figures are merely schematic in nature and function exclusively for understanding the invention. Identical elements are provided with the same reference numerals in the description of the exemplary embodiments.

FIG. 1 shows a schematic view of a measuring arrangement, as it is used in a measuring system according to one exemplary embodiment of the present invention. The depicted exemplary embodiment is suitable for cases in which a measurement is carried out by passing through an egg. The measuring arrangement associated with the measuring system according to the invention has an irradiation unit 10 and a sensor unit 20, between which an egg 50 is arranged for measurement. Sensor unit 20 is thereby arranged on the opposite side of egg 50 when viewed from irradiation unit 10. Sensor arrangement 20 is connected to a spectrometer 31. Power and data connections are provided on spectrometer 31 for connecting to a power and/or data cable, which is schematically shown in FIG. 1 with reference numeral 34.

Irradiation unit 10 is designed to emit radiation in the direction of egg 50, which is arranged in the measuring arrangement. The radiation is electromagnetic radiation, for example, (visible) light, infrared radiation, X-rays or the like. As shown here, an embryo, which is essentially identifiable by blood vessels 52 contained therein, is located underneath an air pocket 51 at the upper end of egg 50 when viewed in the direction of gravity. Irradiation unit 10 is thereby usually formed by a light source in the visible range, for example, a light bulb, an LED, or a xenon arc lamp. However, irradiation units in the meaning of the present invention are all emitters of electromagnetic radiation which are suitable for generating absorption spectra of egg 50. In the exemplary embodiment shown in FIG. 1, irradiation unit 10 has a light guide which functions to guide the electromagnetic radiation, which is emitted by a radiation source (not shown), to egg 50.

Egg 50 faces its blunt end toward irradiation unit 10 and faces its pointed end toward sensor unit 20. Egg 50 is arranged with its pointed end in an optical decoupling element 43, which prevents radiation from directly entering sensor unit 20 without having first passed at least partially through egg 50. Optical decoupling element 43 may thereby have the form of a screen or a brush, which is designed to closely contact the eggshell in order to ensure optimal sealing. This may be preferably enabled, for example, by using elastic or supple materials for producing the decoupling element, or by a brush-like arrangement that contacts egg 50. More preferably, decoupling element 43 is thereby adapted in its basic shape to the accommodation of egg 50.

It is also conceivable, although not necessary, for decoupling element 43 to likewise function as a fixing means and to fix egg 50 in a specific orientation.

On the side facing irradiation unit 10, egg 50 is surrounded by an egg fixing element 101, which likewise functions to fix egg 50 in a predetermined orientation. The orientation of egg 50 may be defined based on an axis that runs through the pointed and blunt ends of egg 50 and to which the shell of egg 50 is substantially rotationally symmetrical.

Radiation emerging from egg 50 is detected by sensor unit 20. An optical waveguide 22 is provided for transmitting the radiation detected at sensor unit 20 to spectrometer 31. It is thereby advantageous, although not necessary, to connect an optical collection element 21 upstream of optical waveguide 22 leading to spectrometer 31 in order to amplify the received signal. Examples of such optical collection elements 21 are collimation lenses, mirrors, or other optical steering elements such as Fresnel lenses.

After the radiation has been recorded, a measured spectrum of the detected radiation is generated by means of spectrometer 31 and forwarded to a data processing unit 33 (not shown) via data cable 34. Data processing unit 33 is communicatively connected to an identification unit 23 (likewise not shown).

Identification data that are uniquely assignable to egg 50 are thereby likewise generated by identification unit 23 and forwarded to data processing unit 33 together with the measured spectra.

In the measuring arrangement of the measuring system, irradiation unit 10 and sensor unit 20 are arranged in such a way that they are aligned relative to at least one reference point of egg 50 for each measurement. Such a reference point is provided, for example, by the center of gravity of the shell of egg 50, which, unlike that of the entire egg, is not influenced by displacements of the embryo. Irradiation unit 10 and sensor unit 20 are preferably identically aligned relative to a reference axis of egg 50 during each measurement. The axis of symmetry, described above, which runs through the poles of the egg, may be used as the reference axis of egg 50. A change in the position of the reference axis of egg 50 is expressed in a tilting of egg 50.

It is possible to tilt the entire arrangement, consisting of egg 50 and the measuring arrangement, while maintaining a constant alignment of irradiation unit 10 and sensor unit 20 relative to said reference points or to the reference axis of egg 50. This enables measurements at different tilt positions of egg 50 while maintaining the same relative arrangement of irradiation unit 10 and sensor unit 20 relative to egg 50. By this means, the embryo may be removed from the illumination field of irradiation unit 10 or the visual field of sensor unit 20 by a controlled tilting in order to, for example, carry out a reference or calibration measurement. Egg 50, together with irradiation unit 10 and sensor unit 20, are therefore always tilted together.

Alternatively, a plurality of measurements may respectively be carried out at different tilt positions of egg 50. In order to determine the current tilt position of egg 50, an angle is thereby determined between a predefined reference axis of egg 50 and a reference axis of the measuring system. The reference axis of the measuring system is also designated to as the axis of reference in the context of the present description. An example of such an axis of reference would be, for example, the connecting line between irradiation unit 10 and sensor unit 20 in FIG. 1. A further example would be an axis of reference in the direction of the gravitational field.

It is thereby not important how the angle between the reference axis and the axis of reference is determined. Mechanical detection is conceivable, by presetting an angle of the egg tray in which the eggs are fixed, or by an optical determination of the angle, for example, by recognizing the egg outline, or by using a gyroscope mounted on the egg tray.

FIG. 2a shows a further exemplary embodiment of the measuring arrangement of a measuring system according to the invention, in which irradiation unit 10 and sensor unit 20 are arranged on the same side of egg 50. In this exemplary embodiment, the sensor unit does not record radiation that has passed through egg 50, but instead radiation that is scattered back in egg 50. The design, with an arrangement of irradiation unit 10 and sensor unit 20 on the same side of egg 50, thereby enables easier tilting of egg tray 40, as the relative position of the irradiation and sensor units 10, 20 to egg 50 automatically remains constant. Such an arrangement is particularly advantageous if the measuring system has a measuring attachment 102, which may be placed on an already existing egg tray 40. In order to minimize the mounting expense and, if necessary, to also ensure optical decoupling from adjacent measuring systems, this type of measuring attachment 102 may have a measuring attachment adapter 103 specifically adapted to egg tray 40.

Measuring attachment adapters 103 are designed in such a way that they engage in an egg tray, or partially accommodate it in order to enable a detachable connection between measuring attachment 102 and egg tray 40, or are placed thereupon.

The measuring attachment adapter additionally functions as an adapter between a plurality of different used egg trays, commonly used in practice, and measuring attachment 102. By this means, the required variance is minimized for a more complex measuring attachment 102.

By providing ventilation openings 106 (not shown here) egg 50 is prevented from insufficient participation in the heat exchange with the incubator air.

This type of measuring attachment may thereby be moved automatically or manually between different egg trays 40 in order to carry out measurements.

In order to introduce a maximum proportion of the radiation, emitted by irradiation unit 10, into the egg, it is advantageous to use beam limiting devices 14, which prevent the light cone, emitted by irradiation unit 20, from spreading too widely. The beam limiting devices may thereby likewise be provided by screens or brushes. They additionally prevent adjacent measuring systems from being disrupted by scattered light.

in order to obtain the maximum information content, even when embryo 52 is not centrally aligned, irradiation unit 10 has multiple irradiation units 10a, as one sees in the exemplary embodiment according to FIG. 2a. This is advantageous in order to optimally acquire the embryo into the visual field in order to ensure a large influence of the embryo on the spectrum.

Since blood vessels 52 often do not grow in the center during the development of egg 50, but instead on one side of the blunt end of egg 50, as shown in FIG. 2a, it is further advantageous to use an irradiation unit 10 with a plurality of radiation sources 10a, in which individual radiation sources 10a may be individually controlled or activated. In the example shown in FIG. 2a, a very low useful signal is to be expected when the left radiation source 10a is activated, while a strong useful signal is to be expected when the right radiation source 10a is activated, which is arranged precisely over blood vessels 52.

When using an irradiation unit 10 with a plurality of individually activatable radiation sources 10a, the generation of a (reference or measurement) spectrum may be carried out in such a way that initial measurements are carried out in which only one radiation source 10a (or only a part of radiation sources 10a) is activated. One or more measurements with a comparatively high useful signal may then be selected from the measurements obtained. If multiple measurements with a comparatively high useful signal are selected, these may be calculated together in a suitable manner, for example averaged, in order to obtain an optimized spectrum. It is likewise possible to calculate the measurement(s) with a comparatively high useful signal with measurements with a comparatively low useful signal in order to improve the signal-to-noise behavior. It is herein conceivable, for example, to divide the measurement with a high useful signal by a measurement with a weak useful signal, or to subtract the measurement with a weak useful signal from the measurement with a high useful signal. The measurement with the comparatively weakest useful signal may be selected as the measurement with a weak useful signal, or multiple measurements with a comparatively weak useful signal may be averaged.

FIG. 2b shows a modification of the exemplary embodiment from FIG. 2a, in which a ring light, which has a plurality of radiation sources 10a arranged in a ring, is used as irradiation unit 10. This represents a structurally simple solution for irradiating egg 50 from a plurality of directions. The diameter of the ring light is selected to be smaller than the diameter of egg 50 at its widest point perpendicular to the axis of symmetry in order to ensure that each radiation source 10a passes radiation through egg 50. The use of a ring LED is particularly preferred. FIG. 2c schematically shows the design of the ring light from FIG. 2b. The ring light has 8 radiation sources 10a, which are provided in a ring-shaped arrangement on the ring light.

FIG. 3 shows a further exemplary embodiment of the present invention, in which the measuring system has multiple measuring arrangements. The respective pairs, made of an irradiation unit with light source 11 and a sensor unit with optical collection element 21, are thereby to be understood as a measuring arrangement. The multiple measuring arrangements may be operated simultaneously to increase the measurement throughput. Collection elements 21 are each provided with optical waveguides 22, which are connected to a spectrometer 31 for detecting the radiation transmitted through eggs 50 and for generating corresponding spectra. Spectrometer 31 is connected to a data processing unit 33, which stores the spectra generated by spectrometer 31 together with identification data, which enables an assignment of the spectra to individual eggs 50 in measured egg tray 40. The data processing unit has connections for power and data cables, which are schematically represented in FIG. 3 and are designated by reference numeral 34. Data processing unit 33 may be connected via connections 34 to a classification unit 35 (not shown in FIG. 3), which is designed to determine the sex of the embryo in individual eggs 50 on the basis of at least one reference spectrum and at least one measured spectrum. Classification unit 35 may also be integrated into data processing unit 33.

Light sources 11 are incorporated into a first measuring arm 60, and collection elements 21 associated with the sensor units are incorporated into a second measuring arm 70. According to this exemplary embodiment, first measuring arm 60 is positioned above and second measuring arm 70 is positioned below an egg tray 40, which is mounted on an egg tray support 41 within an incubator 80. Eggs 50 are thereby located in egg recesses 42 of egg tray 40. However, it is likewise conceivable to attach the measuring arms next to the egg or in any other position, as long as associated irradiation units 10 and sensor units 20 each accommodate one egg between themselves and the relative position of the measuring arrangement to the egg remains constant, or is also rotated along with when the egg is tilted.

As the use of a plurality of light sources, for example LEDs, may lead to increased heat generation, which may have detrimental effects on both the temperature control of the incubator and also on the service life of the light sources, light sources 11 are provided with cooling elements 12. Cooling elements 12 may thereby actively or passively contribute to the cooling. For example, cooling fins are suitable for promoting a faster temperature exchange. The same applies for cooling element 32 of spectrometer 31 used to measure the spectra.

In addition, measuring arms 60, 70 may have ventilation openings in order to guarantee the best possible ventilation around egg 50.

In addition to the beam limiting devices mentioned above, it may be further advantageous to already equip the irradiation unit with an optical steering element 13, for example, an aspherical lens, a Fresnel lens, or the like, in order to couple as much of the radiation emitted by light sources 11 into egg 50 as possible. Furthermore, in the exemplary embodiment shown in FIG. 3, beam limiting devices 14 are provided on the individual lighting units in order to prevent the radiation, emitted by individual light sources 11, from scattering into adjacent measuring arrangements.

Additional measures may be taken in order to keep reciprocal influence between the measuring systems as low as possible. For example, the measuring system may be configured in such a way that a measurement is carried out in multiple, for example, two steps, wherein in a first step, only every second measuring arrangement carries out a measurement, and in a subsequent second step, the remaining measuring arrangements carry out their measurement, so that there is always at least a one egg 50 distance between active measuring arrangements.

In order to easily move the measuring systems back and forth between the multiple egg trays located in a trolley, first measuring arm 60 has a first movement mechanism 61 and second measuring arm 70 has a second movement mechanism 71. These function to shift the position of measuring arms 60, 70 vertically and/or horizontally relative to a measuring column 90, which supports the measuring arms. All egg trays within one trolley may thus be sequentially subjected to a measurement. To transport the egg trays to the measuring system, a mechanism is preferably provided for removing egg trays from the egg trolleys in the incubator, said mechanism may also insert the egg trays back into the egg trolley after the measurement. By this means, a larger installation space may be made available for the measuring unit.

In order to determine the current position of the measuring arrangements and to ensure a unique assignment of the reference and measuring spectra to the different eggs, the measuring system has an identification unit 23. Identification unit 23 is designed to identify an egg tray or a trolley, for example, via attached markers or sensors. In order to guarantee a unique assignment of the measured spectra to eggs 50, identification unit 23 transmits data, for example, an identifier of a trolley, of an egg tray 40, and of a specific egg position within egg tray 40 to data processing unit 33, which stores the identification data of an egg 50 together with the measured reference and/or measured spectra. Individual egg trays 40 are identifiable by way of an egg tray identification feature 44, which may be formed of optically readable codes, for example barcodes or QR codes, or by RFID tags. Identification unit 23 is correspondingly an optical reading device or an RFID reader.

In another embodiment, an identification of trolleys, egg trays 40 or eggs 50 may be carried out by transferring data to data processing unit 33, said data containing information about the current position of measuring arms 60, 70 or movement mechanisms 61, 71. The position of the measuring arrangements may be derived from the position of measuring arms 60, 70 and it may thus be determined which eggs were subjected to measurement.

FIG. 4 shows an external view of a measuring system of the type shown in FIG. 3, with which it is possible to directly measure eggs in an egg tray which is arranged in a trolley. FIG. 4 shows a trolley 80 with a plurality of egg trays 40. The measuring system and trolley 80 are located in an incubator. The incubator wall is designated by reference numeral 104 in FIG. 4.

The measuring system has a first measuring arm 60, a second measuring arm 70 and a measuring column 90. Measuring column 90 is arranged on a column base 91. Column base 91 has a transport device 107 for transporting measuring column 90 between different trolleys 80 within incubator 104. It is also conceivable to use transport device 107 to transport the measuring system between different incubators. In the exemplary embodiment shown in FIG. 4, an evaluation unit 30 is integrated into measuring column 90 which may contain a spectrometer, a data processing unit, and/or a classification unit, or communication means in order to establish a communicative connection to said units.

In order to enable or simplify a localization of the trolleys within the incubator, a trolley positioning device 85 is provided at the floor of an incubator, which ensures that each trolley 80 is positioned at a predetermined location within an incubator. This may be carried out using specific mounting devices or by using markings that allow a user to correctly position trolley 80.

FIG. 5 shows a further exemplary embodiment of the present invention, in which a measuring column 90 is fixed in the incubator such that egg trolleys 80 may be moved to the measuring column. A plurality of measuring arms 94, in which measuring arrangements according to the exemplary embodiments shown in FIG. 1, 2a, 2b or 3 are mounted, are mounted on measuring column 90. The transport of the eggs to the measuring system is carried out by means of a trolley transport device 81, which transports egg trolley 80 as a whole to the measuring column. Trolley transport device 81 may thereby preferably by configured as a robot, which independently moves to the trolley positions and moves trolleys 80 to the fixed measuring system. Trolley transport device 81 has a lower height than the trolley feet 84, so that trolley transport device 81 may move under trolleys 80. An extendable lifting means is provided in the trolley transport device, by means of which egg trolley 80 is lifted and may then be transported.

In order to achieve an adjustment of different tilt angles, trolley transport device 81 may have a trolley tilting device 82 (not shown here), which adjusts the tilt state of egg trays 40. It thereby engages in either an already existing pivot mechanism of egg trolley 80, or it tilts egg trolley 80 as a whole.

FIG. 6 shows the interior of an incubator with multiple measuring systems having one or more measuring arms 60, 70 from a bird's eye view. The incubator is delimited by an incubator wall 104 and an incubator door 105. The measuring systems again have measuring columns 90. Measuring column 90, depicted on the right in FIG. 6, has both a first horizontal linear guide 92 and a second horizontal linear guide 93. These function to adjust the position of measuring arm 94 or measuring arms 60, 70 in a plane orthogonal to measuring column 90. Measuring arm 94 may in turn have measuring arrangements according to the exemplary embodiments shown in FIG. 1, 2a, 2b or 3.

First horizontal linear guide 92 and second horizontal linear guide 93 may thereby be connected to the first movement mechanism of a first measuring arm 60, and a third horizontal linear guide 92 and a fourth horizontal linear guide 93 (which are not visible in FIG. 6, since they are located precisely underneath the first and second horizontal linear guides) may thereby be connected to the second movement mechanism of a second measuring arm 70 in order to enable an adjustment of the position of the measuring system in all three spatial directions. In particular, the first and second measuring arms may thereby be positioned independently from one another, for example to adjust the angle between the measuring system and the egg.

In this way, the measuring system may be moved back and forth between multiple trolleys 80. As shown in FIG. 6, the measuring arms have an identification unit 23, which is designed to read out egg tray identification means 44 on egg trays 40 in order to be able to carry out a unique assignment of egg trays 40 or trolleys 80. A unique position of an egg in a previously identified egg tray 40 or trolley 80 may then be determined from the coordinates of the horizontal linear guide in order to assign the identity of the egg to a measurement.

FIG. 7 again shows one exemplary embodiment of the present invention, in which a measuring column 90 is fixed in the incubator such that trolley 80 may be moved to the measuring column. In order to achieve an adjustment of different tilt angles, trolley transport device 81 engages in a trolley tilting device 82 after it has been brought into position adjacent to trolley foot 84. In the embodiment shown, egg trays 40 are pivoted upward about an axis at the distal end of the egg trays about a tilting mechanism 86 such as a pivot bearing. As the relative alignment of the measuring arrangements to egg 50 is to thereby remain the same, measuring arms 94 are likewise pivotably mounted. The bearing position of measuring arms 94 is coordinated with the position of tilting mechanisms 86, such that the axes of the bearings of the measuring arms and of the tilting mechanisms are superimposed as much as possible when trolley 80 is positioned on measuring column 90 in order to enable a mutual tilting of measuring arms 94 and egg trays 40. It is likewise conceivable to tilt the egg trays to the side or in another way, as long as the relative position to the measuring arrangements is not changed.

FIG. 8 shows a further exemplary embodiment of a measuring arrangement of a measuring system according to the invention of the type shown in FIGS. 2a and 2b, in which irradiation unit 10 and sensor unit 20 are arranged on the same side of eggs 50. The design on the same side thereby enables easier tilting of egg tray 40, as the relative position of the irradiation and sensor units 10, 20 to egg 50 automatically remains constant. Such an arrangement is particularly advantageous for a measuring attachment 102, which may be easily placed on an already existing egg tray 40. In order to minimize the mounting expense, and, if necessary, in order to ensure optical decoupling from adjacent measuring systems, a measuring attachment 102 of this type has measuring attachment adapters 103 specifically adapted to egg tray 40. As shown, it is herein advantageous if the components of the measuring arrangement and egg tray 40 are designed to the largest extent as scaffold-like and/or honeycomb-like, such that the largest possible ventilation openings 106 are formed, via which egg 50 participates in the heat exchange with the incubator air.

FIG. 9 shows another possibility for implementing a tilting mechanism 86 for the egg trays. By hereby raising and lowering egg trays 40 at one end by means of a schematically depicted tilting device 8, a rotation is effected about tilting mechanism 86 designed as a pivot bearing. In particular, if a measuring attachment 102 according to one of the exemplary embodiments shown in FIGS. 2 and 8 is used, care must thereby be taken to ensure that the spacing between the egg trays in the vertical direction is sufficiently large to prevent blocking and, moreover, collisions with the outer wall of trolleys 80.

One further exemplary embodiment is shown in FIG. 10. The arrangement of light sources 11 is designed here such that the beams meet on egg 50 at an angle inclined to the vertical, and thus enter at least partially laterally. The beams emerging from light source 11, emitted substantially radially in all directions, are thereby focused (or bundled) by a first convex lens 111 and by a second convex lens 112, such that resulting light cone 113 has its point on the egg shell. First convex lens and second convex lens 112 are correspondingly arranged in such a way that light emitted from light source 11 enters obliquely into egg 50; preferably at an angle to the vertical of more than 200.

The light emerging from egg 50 then adopts the shape of a diffuse light beam 114, which is in turn bundled by optical collection element 21, in this case a collimating lens, and coupled into optical waveguide 22.

Light sources 11 and optical waveguide 22 are thereby held by a measuring head housing 110. An arrangement is thereby particularly preferred in which light sources 11, as shown in FIG. 2c, are arranged in a ring shape, wherein corresponding light cone 113 is then respectively inclined inwardly toward the center of the ring-shaped arrangement.

One further exemplary embodiment is shown in FIG. 11, which shows an egg trolley 80 in an incubator. Egg trolley 80 thereby stands at incubator wall 104, opposite a mobile egg measuring unit 122, which may be moved as a whole between different incubators. Naturally, it is also possible to locate mobile egg measuring unit 122 behind egg trolley 80 in the incubator. It is only decisive that mobile egg measuring unit 122 is arranged adjacent to egg trolley 80.

Mobile egg measuring unit 122 is thereby positioned such that a tray sled 127 may be moved along a (substantially horizontal) x-axis in order to remove an egg tray 40 from egg trolley 80, and to subsequently deposit it in buffer trays 125 of mobile egg measuring unit 122 provided for this purpose.

Within measuring unit 122, sensor unit 20 is movable along the x-axis, in particular along second horizontal linear guide/measuring arm 93/60 and also along the (substantially vertical) z-axis, so that sensor unit 20 may be moved to each egg of uppermost egg tray 125 in order to carry out a measurement. The movement along the x-axis is thereby carried out along a linear guide or along a second measuring arm 70. As soon as uppermost egg tray 40 has been completely measured, then it may be exchanged with a further tray from egg trolley 80 or from one of buffer trays 125.

In order to prevent a slippage of egg trolley 80, a trolley fixing 120, which detachably connects egg trolley 80 to mobile measuring unit 122, is provided in the exemplary embodiment shown in FIG. 11.

The advantage of the described exemplary embodiment lies in that mobile measuring unit 122 may be “parked” in a closed incubator and egg trays to be measured are then independently transferred into empty buffer trays 125 in order to carry out the measurement. By this means it is prevented that incubator door 105 (not shown) needs to be opened too often, due to which detrimental temperature fluctuations may result. At the same time, mobile measuring unit 122 may be moved back and forth between incubators as needed.

Common to all of the above exemplary embodiments, is that the evaluation unit is designed to output a sex label, on the basis of the measured spectrum of an egg 50, and to determine and output a confidence level of the sex label. The confidence level thereby indicates an estimated probability that the sex label is correct.

On the basis of the confidence level, a decision may additionally be made to subject one specific egg or multiple eggs 50 to further measurements in order to raise the confidence level above a desired value based on the available statistics. At the same time, the observation period for eggs 50 may also be extended, based on the confidence level, in order to increase the same.

A spectrum may thereby be composed of any number of, possibly weighted, individual measurements. The use of multiple measurements, for example, 10 or more, thereby enables better statistics and thus a greater accuracy of the spectrum, which in turn influences the confidence level of the resulting sex labels. In order to keep the duration of a measurement process short, an individual measurement preferably takes no longer than 60 μs, more preferably less than 40 μs, even more preferably less than 20 μs.

According to the present invention, an angle between an absolute reference axis, for example the vertical, and the optical measurement axis is determined during the measurement. The inclination of the measuring axis or of the tilt angle may be determined, for example, by reading out the motor position of the tilting mechanism, using a Hall sensor or potentiometer on the tilting axis of the egg tray, or by measuring the distance between the outer edge of the lowest egg tray and a fixed reference point. Since the embryo always floats at the top in relation to the direction of gravity, the change in angle may be used to influence the location of the embryo within the measuring arrangement.

In particular, for a better evaluation, multiple measurements may be made, which correspond to different positions of the embryo. For example, it is conceivable that, to generate a reference spectrum, the embryo is “tilted out” of the visual field of the sensor unit in order to have a measurement without an embryo as a reference. It is important here that there is a long enough interval between measurements at different tilt angles to enable the embryo to return to a resting position or a state of equilibrium. This prevents the embryo from unintentionally changing position during a measurement. If an irradiation unit with a plurality of radiation sources is used, as described above in relation to FIGS. 2a-2c, further measurements may be carried out using different radiation sources in order to better take into account the different positions of the embryos in the eggs. The concept of multiple measurements with different activated light sources may be combined with the concept of adjusting the tilt angle in order to further improve the quality of the measurements.

A normalization of the measured spectra may be carried out using the stored reference spectra. For example, the signal of the reference spectrum is thereby subtracted from that of a measured spectrum in order to record the actual change and to filter out deviations that arise due to variations in the eggs themselves. In order to guarantee a best possible reference function, it is thereby advantageous to carry out the reference measurement at a very early stage of embryonic development. In particular, it is advantageous for a good reference value to take the reference measurement before incubation begins.

Provision may further be made to calibrate the sensor unit through calibration measurements during operation. For this purpose, reference objects, for example, Teflon blocks are conventionally used, as these influence the spectrum of the radiation source in a known way and thus allow conclusions to be drawn about any measurement errors. In those exemplary embodiments, in which irradiation unit 10 and sensor unit 20 are configured to be mobile, an automation of such calibration measurements may be carried out, in that the measuring system carries out measurements on reference objects at fixed intervals.

It is likewise conceivable to place reference objects on an egg tray 40 in specified trolleys 80 in order to automate the calibration measurements with the aid of trolley transport device 81. In the case of a measuring attachment 102, the sensor unit is calibrated with the aid of a reference tray before placement.

Calibration measurements may likewise comprise measurements in which the sensor unit is intentionally covered in order to check the “dark noise” of the spectrometer.

The wavelength ranges used for sex detection may hereby lie in the absorption range of hemoglobin, i.e., between 500 nm and 900 nm, but are not limited to this.

The evaluation of the measured data may thereby take place directly at data processing unit 33. A final classification is carried out by classification unit 35, which is potentially an external unit.

Classification unit 35 thereby receives the preprocessed measurement data from data processing unit 33. In addition, classification unit 35 accesses externally stored data, which are taken into account when classifying the eggs.

In particular, in the method according to the invention, so-called additional data beyond the pure spectrum may be taken into account during the classification or in the determination of the confidence level. Examples for this include egg size (diameter, height), egg shape, egg weight, egg color, memory time since laying, age of the parent animals, animal breed, origin, egg orientation, orientation of the air bubble in the egg. or evidence of damage to the egg, among others. The additional data is thereby received by data processing unit 33 or classification unit 35 and is included in the determination of the sex or the confidence level. For example, if the egg orientation is suboptimal or the egg is damaged, the confidence level may be reduced or increased. Eggs that are damaged may also generally be assigned to a (non-preferred) sex in order that they may be screened out later.

The additional data may thereby also include further information, for example, the sensor temperature/humidity at at least one point in time, the incubator temperature/humidity at at least one point in time, or fault reports from the incubation process.

In addition, additional data may also contain regulatory requirements or manually defined classification rules and/or screening rules. These allow consideration of the planned maximum or minimum output quantity in the classification. For example, if it becomes apparent that the minimum output quantity of a sex will be undershot, the requirements regarding the confidence level for a classification into said sex are reduced in order to guarantee that there is enough output. In particular, the additional data may thereby also comprise the classification results or confidence level results of other eggs in order to achieve a suitable expected value for the overall output.

The storage of the additional data may thereby be carried out decentrally in a cloud or locally in data processing unit 33.

This enables a precise adjustment of external parameters, for example, the desired sex distribution, minimum quotas for individual sexes, desired output quantity, etc., when deciding whether to screen eggs based on the sex label and confidence level. Other known, estimated or established parameters, for example, the death rate of embryos, may also be considered.

### LIST OF REFERENCE NUMERALS

10 Irradiation unit
11 Light source (LED)
12 (LED) Cooling element
13 Optical steering element (aspherical or Fresnel lens)
14 Beam limiting device
20 Sensor unit
21 Optical collection element (collimating lens)
22 Optical waveguide
23 Identification unit (Identification and position determination unit)
24 Optical decoupling element (sealing ring, thus far only visible in FIG. 2)
30 Evaluation unit
31 Spectrometer
32 Cooling element
33 Data processing unit
34 Connections (power and data cables)
35 Classification unit
40 (Integrated) Egg tray
41 Egg tray support
42 Egg recess
43 Optical decoupling element (rubber coating)
44 Egg tray identification feature (ID tag)
45 Fixing means
50 Egg
52 Blood vessels
60 First measuring arm
61 First movement mechanism
70 Second measuring arm
71 Second movement mechanism
80 Egg trolley
81 Trolley transport device
82 Trolley tilting device
83 Trolley guide element
84 Trolley foot
85 Trolley positioning device
86 Tilting mechanism
90 Measuring column (for measuring arms with linear guides)
91 Column base
92 First horizontal linear guide
93 Second horizontal linear guide
94 Measuring arm
101 Egg fixing element
102 Measuring attachment
103 Measuring attachment adapter
104 Incubator wall
105 Incubator door
106 Ventilation openings
107 Transport means
110 Measuring head housing
111 First convex lens
112 Second convex lens
113 Focused light cone
114 Diffuse light beam
120 Trolley fixing
122 Mobile measuring unit
125 Buffer tray
127 Tray sled

1. Measuring system for non-invasive, preferably automated, sex detection of embryos in an egg (50) in early embryonic development, in particular before the seventh day of incubation, in particular during incubation, in particular in an incubator with at least one egg tray (40) for accommodating a plurality of eggs (50) and at least one egg trolley (80) for holding at least one egg tray (40), having:
at least one irradiation unit (10) for irradiating an egg (50) with electromagnetic radiation;
at least one sensor unit (20) for detecting electromagnetic radiation transmitted through the egg (50);
an evaluation unit (30) with
at least one spectrometer (31) which is designed to receive the radiation transmitted through the egg (50) and to generate a spectrum of the radiation transmitted through the egg (50); and
a data processing unit (33) which is designed to receive spectra generated by the spectrometer (31) and to store them as a reference spectrum or a measured spectrum;
an identification unit (23) for generating identification data, by means of which the spectra generated on an egg (50) can be uniquely assigned to the egg (50);
a classification unit (35);
wherein the data processing unit (33) is designed to store spectra generated by the spectrometer (31) and associated identification data, and wherein the classification unit (35) is designed to determine the sex of the embryo based on at least one reference spectrum and at least one measured spectrum.

2. Measuring system according to claim 1, having an optical decoupling element (43) for optical decoupling of the irradiation unit (10) and the sensor unit (20), said optical decoupling element being preferably designed to contact the egg (50) during a measurement.

3. Measuring system according to claim 1, having means for determining an angle between a reference axis of the egg (50) and a reference axis of the measuring system.

4. Measuring system according to claim 1, wherein the irradiation unit (10) has a plurality of radiation sources (10a) and is preferably designed as a ring light, in which the plurality of radiation sources (10a) is arranged in a ring.

5. Measuring system according to claim 1, wherein the irradiation unit (10) and the sensor unit (20) are arranged and configured in such a way that, during each measurement, they have the same orientation to one another relative to a reference point of the egg (50), for example, to the center of gravity of the shell of the egg (50).

6. Measuring system according to claim 1, having a trolley transport device (81) for transporting an egg trolley (80) with at least one egg tray (40) to at least one irradiation unit (10), said trolley transport device being preferably arranged within the incubator, and/or a trolley positioning device (85) which is suitable for uniquely determining a position of the trolley (80) within the incubator.

7. Measuring system according to claim 1, having transport means (107) for transporting the irradiation unit (10) and the sensor unit (20) to an egg (50), preferably within the incubator, more preferably between different incubators.

8. Measuring system according to claim 1, wherein the trolley transport device (81) has means which are designed to adjust a tilt position of at least one egg (50).

9. Measuring system according to claim 1, having a measuring arm (94) which accommodates the irradiation unit (10) and the sensor unit (20) in such a way that an egg (50) can be positioned for measurement at the irradiation unit (10) and the sensor unit (20).

10. Measuring system according to claim 1, having a first measuring arm (60) which accommodates the irradiation unit (10) and a second measuring arm (70) which accommodates the sensor unit (20), wherein the first measuring arm (60) and the second measuring arm (70) are arranged and designed in such a way that an egg can be positioned for measurement between the irradiation unit (10) and the sensor unit (20).

11. Measuring system according to claim 10, having a measuring column (90) on which the first measuring arm (60) and the second measuring arm (70) are mounted to be movable, wherein the first measuring arm (60) has a first movement mechanism (61) for adjusting a vertical position of the first measuring arm, and wherein the second measuring arm (70) has a second movement mechanism (71) for adjusting a vertical position of the second measuring arm.

12. Measuring system according to claim 11, wherein the first movement mechanism (61) has a first horizontal linear guide (92) and a second horizontal linear guide (93) for adjusting a horizontal position of the first measuring arm (60), and wherein the second movement mechanism (71) has a third horizontal linear guide (92) and a fourth horizontal linear guide (93) for adjusting a horizontal position of the second measuring arm (70).

13. Measuring system according to claim 1, having a measuring attachment (102) which accommodates both the irradiation unit (10) and also the sensor unit (20), so that both the irradiation unit (10) and also the sensor unit (20) can be arranged either above or below the egg tray (80), wherein the measuring attachment (102) is configured by a measuring attachment adapter (103) to be placed on the egg tray (80) during the measurement.

14. Measuring system according to claim 1, comprising at least one ventilation opening (106) which is designed in the measuring attachment (102), in the first measuring arm (60) and/or in the second measuring arm (70), and is configured to ensure ventilation of the egg (50) during a measurement.

15. Measuring system according to claim 1, wherein the evaluation unit (30) is designed to output a sex label and an associated confidence level for each egg (50).

16. Measuring system according to claim 1, having an externally connected data memory, in particular a cloud storage, which is configured to store external parameters, such as mortality rate of the embryos or desired output quantity, measurement and reference spectra, and/or results of the evaluation of the measurement system, and to output to the classification unit (35).

17. Measuring system according to claim 1, comprising fixing means (45) which are designed to prevent a change in the orientation of the egg (50).

18. Measuring system according to claim 1, wherein the classification unit (35) is designed to carry out a classification of an egg (50) according to sex and/or health condition, based on the data from the measuring unit (20) and/or from the externally connected memory, wherein the classification unit (35) is preferably spatially separated from the rest of the evaluation unit (30), and in particular is preferably formed by a software component on an external server, preferably on a cloud server.

19. Method for non-invasive sex detection of an embryo in an egg (50) in early embryonic development, in particular before the seventh day of incubation, in particular during incubation, having the following steps:
Generating at least one reference spectrum before and/or at the beginning of the incubation by means of irradiating the egg (50) with electromagnetic radiation, and detecting radiation that has passed through the egg (50), generating identification data for uniquely identifying the egg (50), and storing the reference spectrum together with the identification data of the egg (50);
Generating at least one measured spectrum during incubation by means of irradiating the egg (50) with electromagnetic radiation, and detecting radiation that has passed through the egg;
Evaluating the measured spectrum using the stored reference spectrum associated with the egg (50) to determine the sex of the embryo.

20. Method according to claim 19, wherein the reference spectrum and/or the measured spectrum are generated from a plurality of individual measurements, preferably from 10 measurements or more, more preferably from 30 measurements or more.

21. Method according to claim 19, wherein an exposure time of less than 60 μs, preferably less than 40 μs, more preferably less than 20 μs is used for generating a measured or reference spectrum.

22. Method according to claim 19, wherein after a measurement is carried out, an angle of the egg (50) to the vertical is changed and a further measurement is carried out using the changed angle of the egg (50) after the egg (50) has reached a state of equilibrium, wherein the irradiation unit (10) and the sensor unit (20) maintain their position relative to the egg (50).

23. Method according to claim 19, wherein generating the at least one reference spectrum and/or generating the at least one measured spectrum comprises a plurality of measurements, in which the egg (50) is irradiated from different directions, preferably from at least 4 different directions, more preferably from at least 6 different directions, more preferably from 8 or more different directions.

24. Method according to claim 23, wherein, during the plurality of measurements while irradiating the egg (50) from different directions, the respective strength of a useful signal is determined in order to find the measurement with a strongest and/or highest useful signal.

25. Method according to claim 19, wherein the measurements with the strongest and/or the weakest useful signal are calculated together in order to obtain an optimized reference spectrum and/or measured spectrum.

26. Method according to claim 19, wherein a normalization of a measured spectrum is carried out based on a stored reference spectrum, wherein the reference spectrum is preferably generated before the incubation, more preferably outside of the incubator, more preferably before reaching the incubation temperature.

27. Method according to claim 19, comprising a calibration measurement for calibrating the sensor unit (20), wherein the calibration measurement is carried out while the sensor is covered and/or on a reference object, for example a Teflon reference block, and wherein the calibration measurement is preferably carried out automatically.

28. Method according to claim 19, wherein, to determine the sex of the embryo, a spectral range is used for the reference and measured spectra in a wavelength range between 520 nm and 580 nm, preferably between 540 nm and 575 nm, more preferably between 520 nm and 680 nm, more preferably between 520 nm and 870 nm.

29. Method according to claim 19, having the inclusion of additional data, in particular from a decentralized data cloud, wherein the evaluation of the measured spectra is carried out taking the additional data into account.

30. Method according to claim 19, wherein a confidence level is assigned to the determination of the sex of the egg (50).

31. Method according to claim 19, wherein the confidence level of the sex determination is taken into account in order to decide whether to screen eggs based on a plurality of adjustable external parameters, such as the incubation cycle-specific death rate of the embryos, the desired output quantity, or sex distribution.

32. Method according to claim 19, wherein an observation period is determined based on the confidence level, and/or further measurements are carried out in order to increase the confidence level.

33. Method according to claim 19, comprising the simultaneous measurement of a plurality of eggs (50), in particular the simultaneous generation of a plurality of reference and/or measured spectra on a plurality of eggs

(50) preferably by means of a plurality of measuring arrangements.

34. Method according to claim 33, wherein the simultaneous measurement of a plurality of eggs (50) is carried out in such a way that the interference between different measuring arrangements is minimized during the generation of the plurality of reference and/or measured spectra.
